(19) United States
(12) Patent Application Publication
(10) Pub. No.: US 2019/0000725 A1
(43) Pub. Date: Jan. 3, 2019

(54) SOOTHING RASH WIPE SYSTEM

(71) Applicant: Rebecca Bailey-Kuykendall, Biglerville, PA (US)

(72) Inventor: Rebecca Bailey-Kuykendall, Biglerville, PA (US)

(21) Appl. No.: 16/022,457

(22) Filed: Jun. 28, 2018

Related U.S. Application Data
(60) Provisional application No. 62/525,931, filed on Jun. 28, 2017.

(51) Int. Cl. A61K 8/02; A47K 10/42

(52) U.S. Cl. CPC A61K 8/0208; A47K 2010/3266; A61K 8/0279; A47K 10/421

(57) ABSTRACT

A soothing rash wipe system including a porous sheet. The porous sheet is impregnated with a diaper rash soothing composition. The diaper rash soothing composition is sodium bicarbonate based and provides soothing effects when applied to skin. The soothing rash wipe system provides a combination of a baby wipe with a baking soda solution to relieve and treat diaper rash, redness, and soreness.

### CROSS REFERENCE TO RELATED APPLICATION

The present application is related to and claims priority to U.S. Provisional Patent Application No. 62/525,931 filed Jun. 28, 2017, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of cloths and wipes and more specifically relates to medicated wipes.

### 2. Description of Related Art

Many parents use diapers. A diaper is a type of underwear that allows the wearer to defecate or urinate without the use of a toilet, by absorbing or containing waste products to prevent soiling of outer clothing or the external environment. Once a diaper has been used, a parent or guardian must then change the diaper to avoid harming the skin of an infant. Babies often suffer from mild to severe diaper rash, resulting in incessant crying, soreness, redness, and pain. Not only is this uncomfortable and traumatic for babies, but it can also be frustrating and stressful on parents. Some parents may rely on diaper rash creams, but such products are messy and take a considerable amount of time to thoroughly apply. Additionally, diaper creams can be highly inconvenient when traveling or away from home. A suitable solution is desired.

U.S. Pat. No. 4,904,524 to Ho-ward J. Yoh relates to a wet wipe. The described wet wipes include a wet wipe comprising a porous sheet impregnated with an aqueous lotion and, concentrated near the surface of the sheet, polymeric beads containing a functional ingredient which is useful for treating the human skin or environmental surfaces and which are characterized in providing controlled release of the functional ingredient, and methods of making said wet wipe.

### BRIEF SUMMARY OF THE INVENTION

In view of the foregoing disadvantages inherent in the known cloths and wipes art, the present disclosure provides a novel soothing rash wipe system. The general purpose of the present disclosure, which will be described subsequently in greater detail, is to provide a combination of a baby wipe with a baking soda solution to relieve and treat diaper rash, redness, and soreness.

A soothing rash wipe system is disclosed herein. The soothing rash wipe system includes a porous sheet. The porous sheet is impregnated with a diaper rash soothing composition. The porous sheet comprises a non-woven fabric such cotton or other suitable material. The diaper rash soothing composition is sodium bicarbonate based and provides soothing effects when applied to skin. The diaper rash soothing composition includes a sodium bicarbonate solution. The porous sheet is formed from at least one layer and is pre-moistened with the sodium bicarbonate solution. The sodium bicarbonate solution is formulated to sooth and prevent diaper rashes. The porous sheet may be provided as a perforated multi-count roll. The multi-count roll may be stored within a housing. The housing includes an opening or slit for feeding the multi-count roll therethrough. The porous sheet is dispensed from the housing for use.

A method of soothing a diaper rash using soothing rash wipe system is also disclosed herein including the steps of providing a porous sheet impregnated with a diaper rash soothing composition and wiping and treating skin of a baby using the porous sheet.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a soothing rash wipe system, constructed and operative according to the teachings of the present disclosure.

FIG. 1 is a perspective view of the soothing rash wipe system during an ‘in-use’ condition, according to an embodiment of the disclosure.

FIG. 2 is a perspective view of the soothing rash wipe system of FIG. 1, according to an embodiment of the present disclosure.

FIG. 3 is a perspective view of the soothing rash wipe system of FIG. 1, according to an embodiment of the present disclosure.

FIG. 4 is a perspective view of the soothing rash wipe system of FIG. 1, according to an embodiment of the present disclosure.

FIG. 5 is another perspective view of the soothing rash wipe system of FIG. 1, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to cloths and wipes and more particularly to a soothing rash wipe system as used to improve the treatment and promote the prevention of diaper rashes.

Generally, the present invention soothes and comforts babies, while making it easier for parents to care for their children. It combines a baby wipe with a baking soda solution to relieve and treat diaper rash, redness, and soreness. This eliminates the mess and hassle associated with traditional diaper rash creams. This further helps calm crying, fussy babies. The present invention provides parents with comfort and peace of mind. Soothing rash wipe system treats diaper rash proactively with ease and convenience, resulting in happier babies and parents. This innovative product may comprise a disposable rectangular or circular wipe, made from cotton or other suitable material. The wipe can be thicker than a traditional baby wipe and can be pre-moistened with a baking soda solution. The baking soda can instantly soothe a baby's sore bottom on contact and, over time, eliminate the rash. Further, the wipe can be offered in multi-count perforated rolls, contained within a sturdy plastic housing. The roll of wipes can feed up through a slit in the top of the box, allowing for parents to easily pull up on the roll and tear off a single wipe.

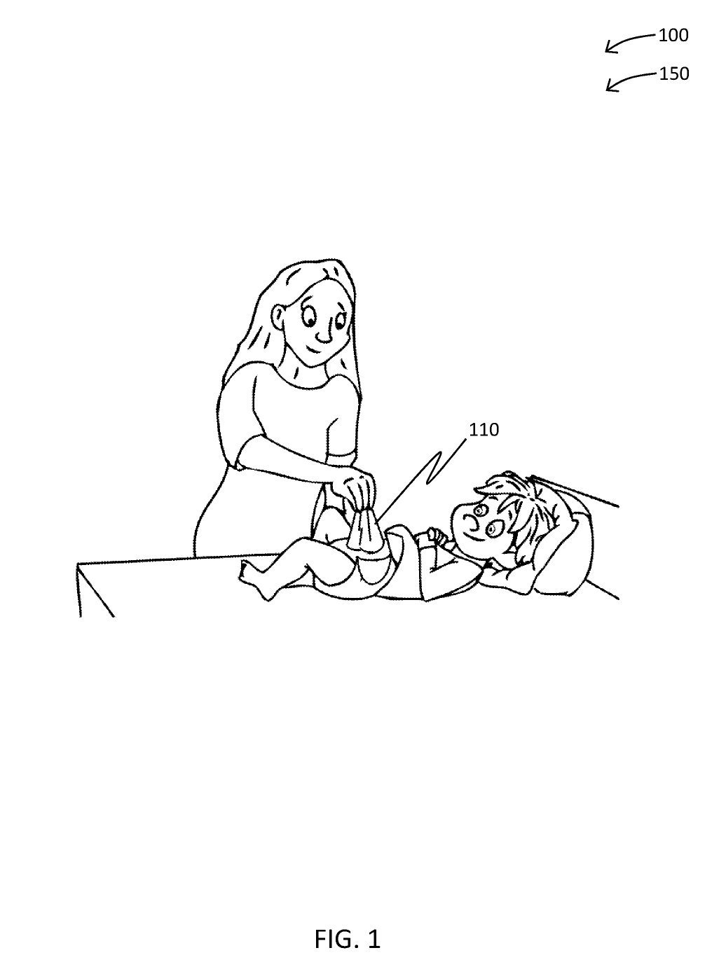

Referring now more specifically to the drawings by numerals of reference, there is shown in FIGS. 1-4, various views of a soothing rash wipe system 100. FIG. 1 shows a soothing rash wipe system 100 during an ‘in-use’ condition 150, according to an embodiment of the present disclosure. As illustrated, the soothing rash wipe system 100 may include a porous sheet 110. The porous sheet 110 is impregnated with a diaper rash soothing composition. The diaper rash soothing composition is preferably sodium bicarbonate based and provides soothing effects when applied to skin. The sodium bicarbonate, prevents and treats diaper rash symptoms. Other solutions are available.

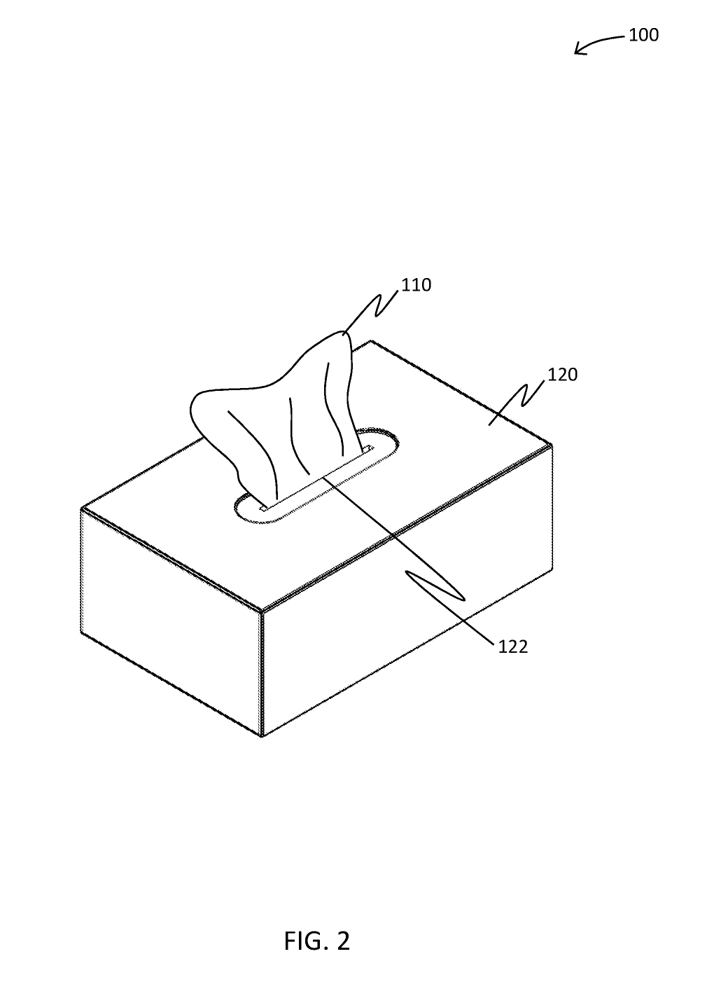

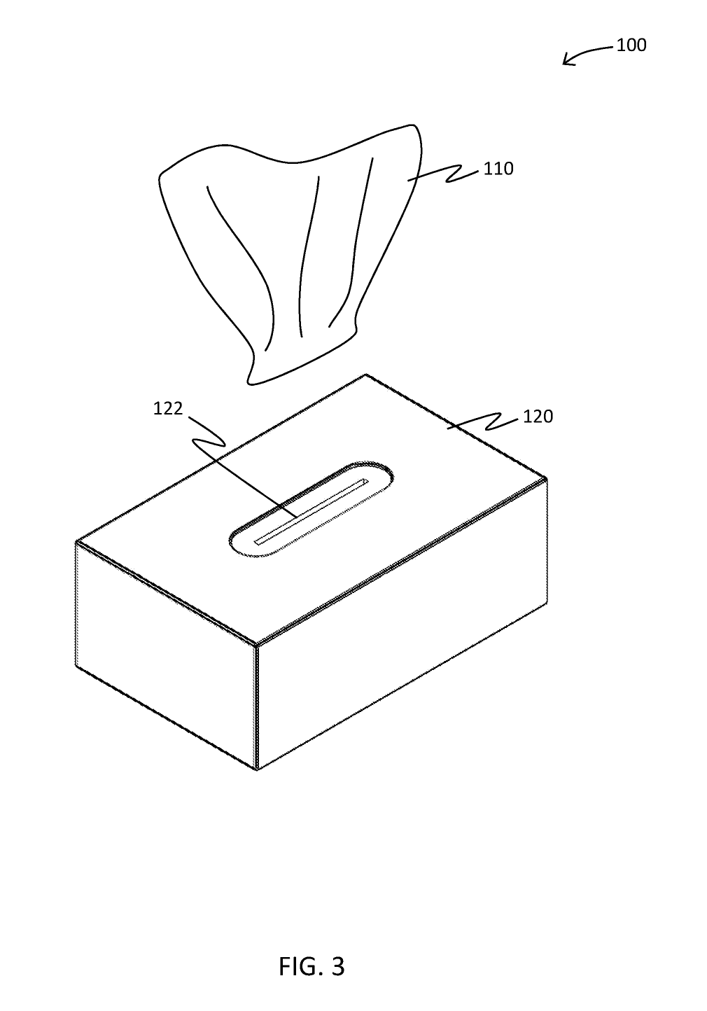

FIGS. 2-3 show perspective views of the soothing rash wipe system 100 of FIG. 1, according to an embodiment of the present disclosure. As above, the soothing rash wipe system 100 may include the porous sheet 110 impregnated with a diaper rash soothing composition. The diaper rash soothing composition includes a sodium bicarbonate solution. The porous sheet 110 includes a moisturizer allowing for wiping and cleaning of an infants between diaper changes. The porous sheet 110 is pre-moistened with the sodium bicarbonate solution. The sodium bicarbonate solution is formulated to sooth and prevent diaper rashes. The porous sheet 110 can be thicker than a traditional baby wipe (at least 0.5 millimeters). The porous sheet 110 is disposable and comprises a non-woven fabric such as cotton or other suitable materials. The porous sheet 110 is used to treat and prevent diaper rashes and soothe skin.

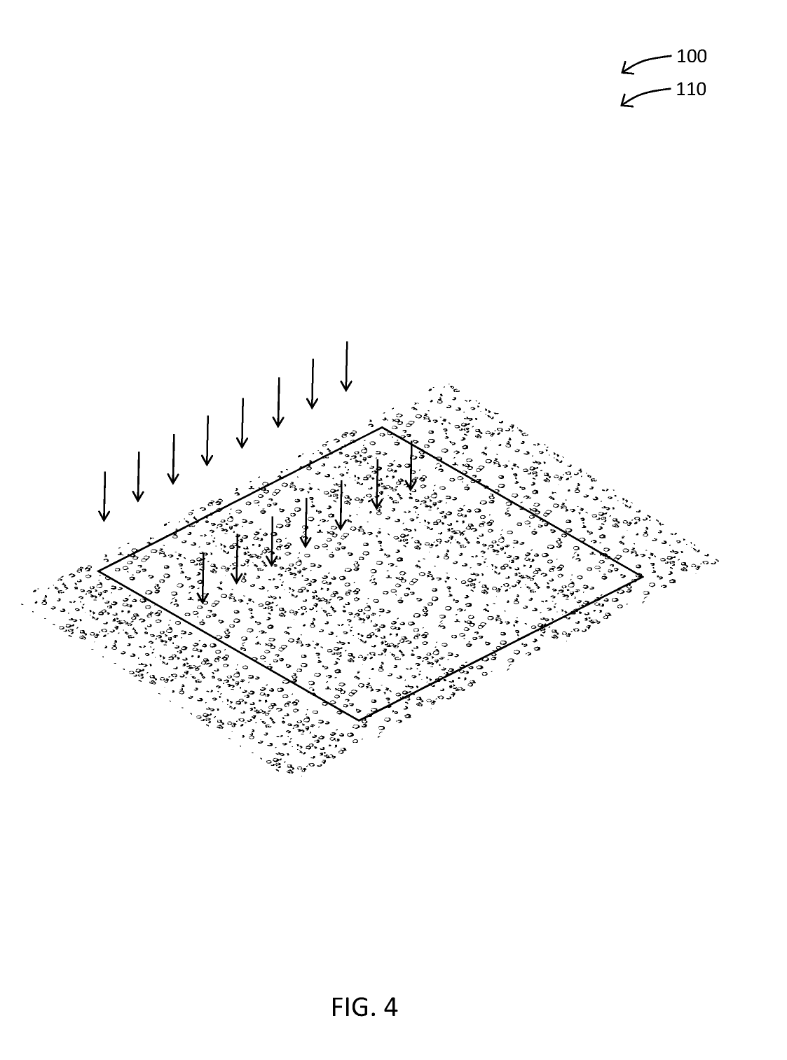

Referring now to FIG. 4 showing a perspective view of the soothing rash wipe system 100 of FIG. 1, according to an embodiment of the present disclosure. As above, the soothing rash wipe system 100 may include the porous sheet 110 provided in a multi-count roll. In a preferred embodiment, the multi-count roll is perforated. The multi-count roll may be stored within a housing 120. The housing 120 includes an opening 122 or slit for feeding the multi-count roll therethrough. The porous sheet 110 is dispensed from the housing 120 for use. The porous sheet 110 may be rectangular, circular or other suitable shapes.

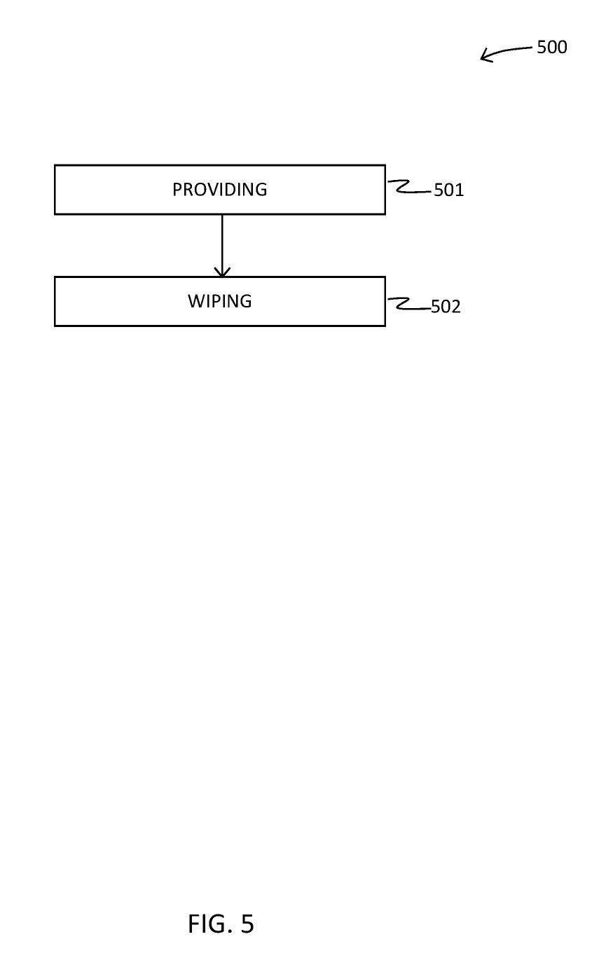

FIG. 5 is a flow diagram illustrating a method for soothing a diaper rash using soothing rash wipe system 500, according to an embodiment of the present disclosure. In particular, the method for soothing a diaper rash using soothing rash wipe system 500 may include one or more components or features of the soothing rash wipe system 100 as described above. As illustrated, the method for soothing a diaper rash using soothing rash wipe system 500 may include the steps of: step one 501, providing a porous sheet 110 impregnated with a diaper rash soothing composition; and step two 502, wiping and treating skin of a baby using the porous sheet 110.

It should be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for soothing a diaper rash using soothing rash wipe system, are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A soothing rash wipe system comprising:
a porous sheet;
wherein said porous sheet is impregnated with a diaper rash soothing composition; and
wherein said diaper rash soothing composition is sodium bicarbonate based and provides soothing effects when applied to skin.

2. The soothing rash wipe system of claim 1, wherein said diaper rash soothing composition includes a sodium bicarbonate solution.

3. The soothing rash wipe system of claim 2, wherein said porous sheet is pre-moistened with said sodium bicarbonate solution.

4. The soothing rash wipe system of claim 3, wherein said sodium bicarbonate solution is formulated to sooth and prevent diaper rashes.

5. The soothing rash wipe system of claim 1, wherein said porous sheet comprises a non-woven fabric.

6. The soothing rash wipe system of claim 5, wherein said non-woven fabric includes cotton.

7. The soothing rash wipe system of claim 1, wherein a thickness of said porous sheet is at least 0.5 millimeters.

8. The soothing rash wipe system of claim 1, wherein said porous sheet is provided in a multi-count roll.

9. The soothing rash wipe system of claim 8, wherein said multi-count roll is perforated.

10. The soothing rash wipe system of claim 9, wherein said multi-count roll is stored within a housing.

11. The soothing rash wipe system of claim 10, wherein said housing includes an opening for feeding said multi-count roll therethrough.

12. The soothing rash wipe system of claim 11, wherein said porous sheet is dispensed from said housing for use.

13. The soothing rash wipe system of claim 1, wherein said porous sheet is rectangular in shape.

14. The soothing rash wipe system of claim 1, wherein said porous sheet is circular in shape.

15. The soothing rash wipe system of claim 1, wherein said porous sheet is disposable.

16. The soothing rash wipe system of claim 1, wherein said porous sheet is formed from at least one layer.

17. The soothing rash wipe system of claim 1, wherein said porous sheet includes a moisturizer.

18. A soothing rash wipe system, the soothing rash wipe system comprising:
a porous sheet;
wherein said porous sheet is impregnated with a diaper rash soothing composition; and
wherein said diaper rash soothing composition is sodium bicarbonate based, and provides soothing effects when applied to skin;
wherein said diaper rash soothing composition includes a sodium bicarbonate solution;
wherein said porous sheet is pre-moistened with said sodium bicarbonate solution;
wherein said sodium bicarbonate solution is formulated to sooth and prevent diaper rashes;
wherein said porous sheet comprises a non-woven fabric;
wherein said non-woven fabric includes cotton;
wherein said porous sheet is provided in a multi-count roll;
wherein said multi-count roll is perforated;
wherein said multi-count roll is stored within a housing;
wherein said housing includes an opening for feeding said multi-count roll therethrough;
wherein said porous sheet is dispensed from said housing for use;
wherein said porous sheet is disposable;
wherein said porous sheet is formed from at least one layer; and
wherein said porous sheet includes a moisturizer.

19. A method of soothing a diaper rash using soothing rash wipe system, the method comprising the steps of:
providing a porous sheet impregnated with a diaper rash soothing composition; and
wiping and treating skin of a baby using said porous sheet.
